(19) United States
(12) Patent Application Publication
(10) Pub. No.: US 2025/0235571 A1
(43) Pub. Date: Jul. 24, 2025

(54) AROMA AND LIGHTING EMITTING SYSTEM

(71) Applicant: DONGGUAN YIH TEH ELECTRIC PRODUCTS CO., LTD., Dongguan City (CN)

(72) Inventor: Ming Jen HSIAO, Miaoli County (TW)

(21) Appl. No.: 18/418,965

(22) Filed: Jan. 22, 2024

(51) Int. Cl. A61L 9/03; F21V 3/06; F21V 33/00

(52) U.S. Cl. CPC A61L 9/03; F21V 3/06; F21V 33/0044; A61L 2209/11; A61L 2209/133; A61L 2209/15

(57) ABSTRACT

An aroma and lighting emitting system that includes a detachable aromatic substance container and an aroma warmer that uses a hollow heating element to heat a thermal conduction device inside a light-transmissive shell with light holes, which to heat aromatic substances of the detachable aromatic substance container for producing fragrance, and a light source which is inside the light-transmissive shell with light holes can emit light through the shell and light holes to provide a lighting effect, and to further release lighting and fragrance in a space to create a result of a visual-active and olfactory-active atmosphere within an environment.

### BACKGROUND OF THE INVENTION

### 1. Field of the Invention

The present invention relates to an aroma and lighting emitting system.

### 2. Description of the Related Art

In the prior art of aroma warmer designs, candle warmers are made by ceramics or a low thermal conductivity material with a great volume defined inside and with a heater burned scented candle releasing fragrance without a light emitting effect. For example, from the present inventor, such as U.S. Pat. No. 7,133,605 B2 and U.S. patent application Ser. No. 16/828,043, a container is used to transmit a heated source to an aromatic substance. The aromatic substance is heated in the middle and the other part of the aromatic substance is far away from the heated source. The fragrance is only generated in the middle part of the aromatic substance, so it is less easy to produce a sufficient amount of fragrance instantly, and you need to wait for a long time to smell the fragrance. Also, the heated source is concentrated in the central part of the aromatic substance, so it is difficult to completely vaporize the aromatic substance, and the aromatic substance residual is often formed into waste.

In the existing aroma warmer designs, for example, a heating container of a candle warmer houses a fixed aromatic substance. For example, a metal container fixed to the aroma warmer. The interior of the metal container is filled with an aromatic substance, so the use of aromatic substances and the replacement of aromatic substances are limited.

### SUMMARY OF THE INVENTION

The present invention has been accomplished under the circumstances in view, in some embodiments of the present invention disclose an aroma and lighting emitting system, which provides lighting effect and fragrance in a space to create a result of a olfactory-active and visual-active atmosphere within an environment;

The following embodiments of the present invention is to provide an aroma and lighting emitting system comprising:
an aroma warmer, said aroma warmer comprising a connection device, a hollow heating element, a thermal conduction device, a shell, a housing, a control circuit board with an illumination device, and a base, said shell having a first top opening and an opposing first bottom opening, said housing having a second top opening and an opposing second bottom opening, said base being fastened to said second bottom opening of said housing, said connection device being mounted inside said housing and having one end thereof facing toward said second top opening and fastened to a bottom of said thermal conduction device, said hollow heating element surrounding said connection device, said hollow heating element having an opposing top side abutted against a bottom side of said thermal conduction device.

The shell can detachably assemble on the housing, the shell defines an interior volume that is adapted for a detachable aromatic substance container being heated by the thermal conduction device from the hollow heating element to release scent through the first top opening.

Said connection device has an opposing end fastened to the base; said shell is provided with a plurality of light holes for radiating the light from the control circuit board with an illumination device; whereby provides lighting effect and fragrance in a space to create a result of olfactory-active and visual-active atmosphere within an environment.

It is some other embodiments of the present invention to provide an aroma and lighting emitting system comprising an aroma warmer, which allows the aromatic substance container from the aroma warmer to be removably and detachably replaced easily. Accordingly, the multiple choices of aromatic substance flavors are not limited.

In some embodiments of the present invention to provide an aroma and lighting emitting system comprising an aroma warmer, the hollow heating element is an annular heating element or a similar annular heating element. The annular heating element can distribute the heated source between the center and surroundings of the thermal conduction device. The feature of the annular heating element is that instead of focusing on the heated source in the center, it will let the heated source scattered around. In this way, the annular heating element can heat the thermal conduction device evenly so the thermal conduction device can evenly transfer heat to the aromatic substance container, which enables the aromatic substances to be completely vaporized without leaving a residue.

The present invention eliminates the problems of limiting heated source power and poor heating of aromatic substances of conventional aroma warmers. The heated sources of the conventional aroma warmers are provided to focus on the center of the aromatic substance. Due to the limitation of the aromatic substance, the heating effect of the conventional aroma warmers is poor, so after the aroma warmer is powered on, you also need to wait for a period of time to allow enough fragrance to spread into the space. The annular heating element of the aroma warmer and lighting emitting system of the present invention disperses the heated source to evenly heat the aromatic substance, improving the problem that the heated source of the conventional heating element is concentrated in the center of the aromatic substance. At the same time, the aroma warmer and lighting emitting system of the present invention can also rapidly generate an aromatic scent in the air by using a dispersed heated source of the annular heating element.

### BRIEF DESCRIPTION OF THE DRAWINGS

FIG. 1 is a perspective view of an aroma and lighting emitting system in accordance with the present invention.

FIG. 2 is an exploded view of the aroma warmer using a detachable aromatic substance container of the aroma and lighting emitting system in accordance with the present invention.

FIG. 3 is a cross-sectional view of the aroma warmer using a detachable aromatic substance container of the aroma and lighting emitting system in accordance with the present invention.

### DETAILED DESCRIPTION OF THE PREFERRED EMBODIMENT

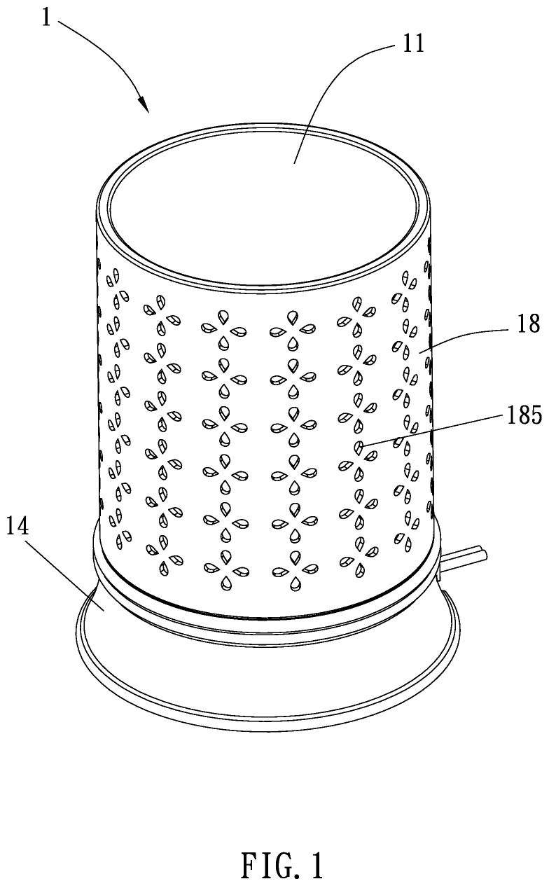

Referring to FIGS. 1-3, an aroma and lighting emitting system comprising:
an aroma warmer 1, said aroma warmer 1 comprising a connection device 13, a hollow heating element 16, a thermal conduction device 17, a shell 18, a housing 14, a control circuit board with illumination device 187, and a base 19, said shell 18 having a first top opening 181 and an opposing first bottom opening 183, said housing 14 having a second top opening 141 and an opposing second bottom opening 143, said base 19 being fastened to said second bottom opening 143 of said housing 14, said connection device 13 being mounted inside said housing 14 and having one end thereof facing toward said second top opening 141 and fastened to a bottom of said thermal conduction device 17, said hollow heating element 16 surrounding said connection device 13, said hollow heating element 16 having an opposing top side abutted against a bottom side of said thermal conduction device 17;
the shell 18 can detachably assemble on the housing 14, the shell 18 defines an interior volume that is adapted for a detachable aromatic substance container 11 (for example, aromatic wax and scented candles) being heated by the thermal conduction device 17 from the hollow heating element 16 to release scent through the first top opening 181.

The base 19 comprises a stud 191, said stud 191 having a hole 193 in the center; said connection device 13 has an opposing end fastened to the hole 193 of said stud 191 of said base 19; said shell 18 is provided with a plurality of light holes 185 for radiating the light from the control circuit board with illumination device 187; whereby provides lighting effect and fragrance in a space to create a result of olfactory-active and visual-active atmosphere within an environment.

In some embodiments of the present invention, wherein said aroma warmer 1 is adapted for heating said aromatic substance of aromatic substance container 11, said aromatic substance container 11 can be removably touched on said thermal conduction device 17.

In some embodiments of the present invention, wherein said lighting holes 185 are continuously assembled with four diamond-shaped holes for radiating the light from the control circuit board with illumination device 187 to form a star-light radiation effect.

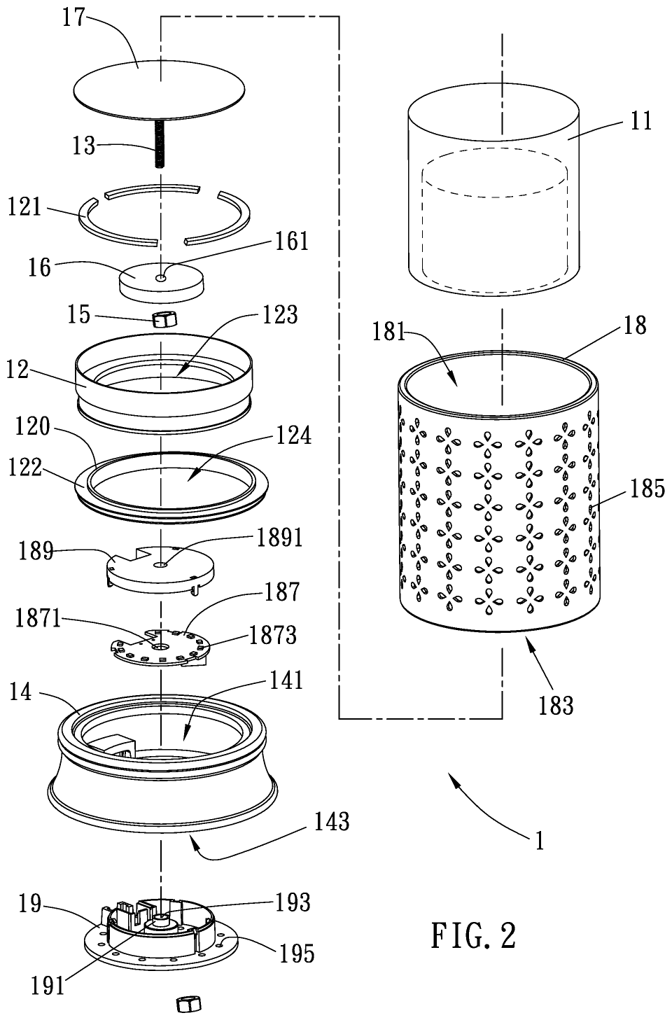

Referring to FIGS. 1 and 2, in some embodiments of the present invention, wherein said shell 18 is made from light-transmissive or semi-light-transmissive materials, for example, semi-light-transmissive or light-transmissive ceramics, plastic, silicone, glass, rubber, or polymer, in some instances, the shell 18 is made from semi-light-transmissive ceramics, light-transmissive ceramics, semi-light-transmissive plastic, light-transmissive plastic, semi-light-transmissive glass or light-transmissive glass which is a light permissive hollow wall of shell 18, the light source 1873 can emit light through the shell 18 and the lighting hole 185 is provided for creating a lighting effect, whereby provides lighting effect in a space to create a result of visual-active atmosphere within an environment;

Referring to FIGS. 1 and 2, in some embodiments of the present invention, the aroma warmer 1 further comprises a light guide loop 12, the light guide loop 12 made from light-transmissive or semi-light-transmissive materials which can be in colors or transparent or semi-transparent; the control circuit board with illumination device 187 comprises a light source 1873 is electrically connected with a power unit (not shown in the figures), the light guide loop 12 is configured at the outer of the thermal conduction device 17 which is inside the shell 18, hereby, the light source 1873 emitting the light in the light guide loop 12, and evenly disperses lighting effect with or without colors inside the shell 18 with halo effect through the lighting holes 185 and the semi-light-transmissive ceramics of shell 18 or the light-transmissive ceramics of shell 18, which is a thermally conductive and light permissive hollow wall of shell 18 whereby provides lighting effect in a space to create a result of visual-active atmosphere within an environment.

Said light guide loop 12 including an inner flange 123, the inner flange 123 formed in the inner of the light guide loop 12, and the thermal conduction device 17 arranged on the inner flange 123.

Referring to FIGS. 1-3, in some embodiments of the present invention, the aroma and lighting emitting system, wherein aroma warmer comprises an insulating unit 173 placed between the light guide loop 12 and the thermal conduction device 17. In the figures of the embodiments of the present invention, the insulating unit 173 disposed on inner flange 123 of the light guide loop 12, the thermal conductive device places on the insulating unit 173 selected from silicone, rubber or plastic.

Referring to FIGS. 1 and 2, in some embodiments of the present invention, the aromatic substance container and the shell 18 can be replaced easily, said aroma warmer comprising a support loop 120 and multiple tabs 122 formed around a peripheral side of the support loop 120, the support loop 120 arranged around the light guide loop 12 when the shell 18 arranged on the housing 14 and cover the light guide loop 12 and support loop 120, the inner wall of the shell 18 is removably retained with the multiple tabs 122 of the support loop 120, wherein the multiple tabs 122 are flexible, when the shell 18 cover on housing 14, the inner wall of the shell 18 forces the multiple tabs 122 to flexibly deform, so that the multiple tabs 122 urges the support loop 120 to abut against the inner wall of the shell 18, furthermore, the shell 18 removable from the support loop 120 and the housing 14, which further allows the aromatic substance container 11 from the aroma warmer 1 to be removably and detachably replaced easily. Accordingly, the multiple choices of aromatic substance flavors are not limited.

Referring to FIGS. 1 and 2, in some embodiments of the present invention, wherein comprising an aroma substance container 11 disposed in the light guide loop 12 inside the shell 18, the aroma substance container 11 made from light-transmissive or semi-light-transmissive materials which can be in colors or transparent or semi-transparent; the light source 1873 emitting the light in the light guide loop 12 and evenly disperse lighting with or without colors inside the shell 18, and the lighting reflects in the aroma substance container 11 producing lighting effect with or without colors through the whole shell 18 and the lighting holes 185 whereby provides lighting effect in a space to create a result of a visual-active atmosphere within an environment.

In some embodiments of the present invention, the aroma and lighting emitting system: a user can fill the desired aromatic substances into a new aromatic substance container 11 for heating by the thermal conduction device 17 from the hollow heating element 16 to produce fragrance. These aromatic substances can be solid or liquid aromatic substances with special flavors, for example, essential oils, and aromatic waxes or scented candles hereby are to be interpreted very broadly so as to include all bodies of scented hydrocarbon material both liquid and solid, whether they comprise a wick or not.

Referring to FIGS. 1-3, in some embodiments of the present invention, the aroma and lighting emitting system, wherein aroma warmer comprises a fixing device set 15, a transparent lighting case 189, the transparent lighting case 189 having a third hole 1891; the control circuit board with illumination device 187 having a fourth hole 1871, the hollow heating element 16 having connection hole 161; the transparent lighting case 189 cover the control circuit board with illumination device 187 for protection, the connection device 13 connects through the connection hole 163, the fixing device set 15, the third hole 1891, and the fourth hole 1871; the fixing device set 15 supports the bottom side of the hollow heating element 16, whereby the light source 1873 can emit light through the transparent lighting case 189 to light guide loop 12, the light guide loop 12 receives the light and forms a lighting effect to emit the lighting effect through the shell 18 and the lighting hole 185, whereby providing lighting effect in a space to create a result of visual-active atmosphere within an environment.

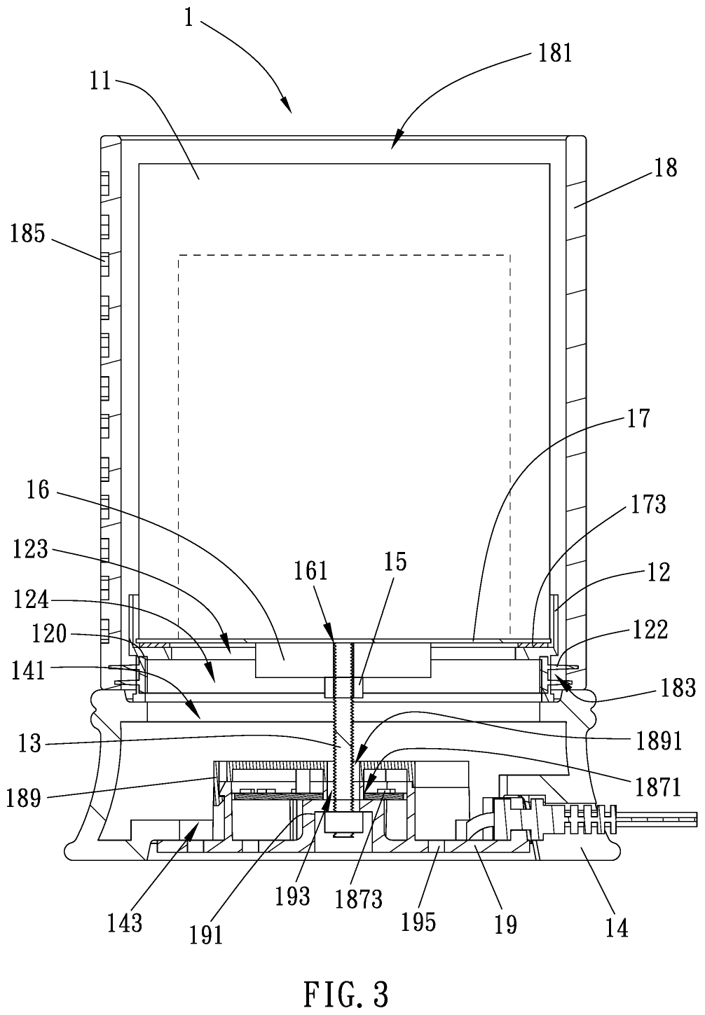

Referring to FIGS. 1-3, in some embodiments of the present invention, the aroma and lighting emitting system, wherein the hollow heating element 16 can be an annular heating element or similar annular heating element, for example, a hollow plum-blossom-shaped heating element, or a hollow irregular annular-shaped heating element (not shown in the figures). In the embodiment shown in FIGS. 2 and 3, the thermal conduction device 17 can be a metal round plate or a similar metal round plate; the annular heating element 16 or similar annular heating element can distribute the heated source between the center and surrounding of the metal round plate or a similar metal round plate.

The feature of the annular heating element 16 is that instead of focusing the heated source in the center, it will let the heated source be scattered around. In this way, the annular heating element 16 can heat the metal round plate evenly so that the thermal conduction device 17 can evenly transfer heat to the aromatic substances in the aromatic substance container 11 to completely heat and vaporize the aromatic substances. The annular heating element 16 is electrically connected to a power source to generate heat for heating the metal round plate. The structure of the thermal conduction device 17 does not concentrate the heated source, and it can evenly heat the aromatic substance container 11 to quickly generate fragrance in the space and completely heat and vaporize the aromatic substances in the aromatic substance container 11, avoiding waste of aromatic substances.

The beneficial effects of the present invention are outlined hereinafter, the annular heating element can distribute the heated source between the center and surrounding of the thermal conduction device 17. The feature of the hollow heating element is that instead of focusing the heated source in the center, it will let the heated source be scattered around. In this way, the hollow heating element 16 can heat the thermal conduction device 17 evenly so that the thermal conduction device 17 can evenly transfer heat to the aromatic substances in the aromatic substance container 11 to completely heat and vaporize the aromatic substances without leaving a residue in the aromatic substance container to create a result of olfactory-active atmosphere within an environment.

According to some embodiments of the present invention, the aromatic substance container 11 can be detachably attached to the top side of the metal round plate (thermal conduction device 17), so that the heated source can be transferred to the aromatic substance in the aromatic substance container 11, causing generation of fragrance. The aromatic substance container 11 selectively uses the detachable semi-light-transmissive or light-transmissive materials, for example, glass, semi-light-transmissive ceramic aromatic substance container 11 or light-transmissive ceramic aromatic substance container 11 . . . etc. When the aromatic substance container 11 is selected, the new aromatic substance container 11 can provide different aromatic substances in the aroma warmer 1 of the aroma and lighting emitting system. The aroma and lighting emitting system of the present invention highlights the convenience and selectivity of the use of aromatic substances.

Referring to FIGS. 1-3, in some embodiments of the present invention, the aroma and lighting emitting system, wherein the aroma warmer comprises an insulating unit 173 placed between the light guide loop 12 and the thermal conduction device 17. In the figures of the embodiments of the present invention, the insulating unit 173 disposed in inner flange 123 of the light guide loop 12, the thermal conductive device placed on the insulating unit 173 selected from silicone, rubber or plastic.

Referring to FIGS. 1-3, in one embodiment of the present invention, the thermal conduction device 17 is, for example, any one of metal, ceramic, glass, etc. in another embodiment of the present invention, the thermal conduction device 17 is, for example, iron, aluminum, copper, or an alloy made of metal, and has good thermal performance.

Referring to FIGS. 1-3, according to some embodiments of the present invention, the thermal conduction device 17 includes, for example, a metal sheet or a ceramic sheet. In the embodiment shown in FIGS. 2-3, the thermal conduction device 17 is a metal sheet, and the metal sheet can be, for example, an iron sheet.

The aromatic substance container 11 can be detachably attached to the top side of the metal sheet (thermal conduction device 17), so that the heated source can be transferred to the aromatic substance in the aromatic substance container 11, causing generation of fragrance. The user can selectively use the detachable glass or light-transmissive ceramic aromatic substance container 11. When the aromatic substance container 11 is selected, the user can replace different aromatic substances in the aroma warmer 1 of the aroma and lighting emitting system. The aroma and lighting emitting system of the present invention highlights the convenience and selectivity of the use of aromatic substances and solves the problem of the limitation of the function of conventional aroma warmers.

Referring to FIGS. 1-3, in one embodiment of the present invention, the shell 18 is light-transmissive or semi-light-transmissive materials, such as light-transmissive ceramic, plastic, glass, colored plastic film silicone, or the shell 18 can be provided with light holes. The control circuit board with illumination device 187 further comprises a light source 1873 mounted in the control circuit board with illumination device 187 inside the shell 18. The light source 1873, such as an LED or a light bulb, connected to a power source (not shown in figures) to emit light radiation through the shell 18 and the light holes to produce a lighting effect.

In one embodiment of the present invention, the shell 18 is a light-transmissive or semi-light-transmissive ceramics for creating a halo effect from a LED (light source 1873), and the light radiates through the light holes 185, thereby creating a lighting effect.

Referring to FIGS. 1-3, in some embodiments of the present invention, the connection device 13 is, for example, a screw rod. The fixing device set 15 is an anti-loosening nut. Thus, the anti-loosening nut maintains and keeps the hollow heating element 16 in close contact with the thermal conduction device 17 to sustain the heat from the hollow heating element 16 to the thermal conduction device 17.

Referring to FIGS. 1-3, in one embodiment of the present invention, the aromatic substance 122 is selected from any one, for example, including scented wax (aromatic wax), scented candles, wax melts, fragrance blends, or essential oil.

After the aromatic substance is used up, the aromatic substance container 11 can be changed easily. Thus, the user's hands will not be contaminated by the aromatic substance. In addition, the user does not necessary to clean the thermal conduction device 17 of the aroma warmer 1.

1. An aroma and lighting emitting system comprising:
an aroma warmer, said aroma warmer comprising a connection device, a fixing device set, a hollow heating element, a thermal conduction device, a shell, a housing, a control circuit board with an illumination device, and a base, said shell having a first top opening and an opposing first bottom opening, said housing having a second top opening and an opposing second bottom opening, said base being fastened to said second bottom opening of said housing, said connection device being mounted inside said housing and having one end thereof facing toward said second top opening and fastened to a bottom of said thermal conduction device, said hollow heating element surrounding said connection device, said hollow heating element having an opposing top side abutted against a bottom side of said thermal conduction device;
the shell can detachably assemble on the housing, the shell to define an interior volume that is adapted for a detachable aromatic substance container being heated by the thermal conduction device from the hollow heating element to release scent through the first top opening;
said connection device having an opposing end fastened to the base; said shell is provided with a plurality of light holes for radiating the light from the control circuit board with the illumination device.

2. The aroma and lighting emitting system as claimed in claim 1, wherein said aromatic substance container can be removably touched on said thermal conduction device.

3. The aroma and lighting emitting system as claimed in claim 1, wherein said lighting holes are continuously assembled with four diamond-shaped holes for radiating the light from the control circuit board with the illumination device to form a star-light radiation.

4. The aroma and lighting emitting system as claimed in claim 1, wherein said shell is made from light-transmissive or semi-light-transmissive materials.

5. The aroma and lighting emitting system as claimed in claim 4, wherein the shell is semi-light-transmissive ceramics, light-transmissive ceramics, semi-light-transmissive plastic, light-transmissive plastic, semi-light-transmissive glass or light-transmissive glass.

6. The aroma and lighting emitting system as claimed in claim 4, wherein the aroma warmer further comprises a light guide loop, the light guide loop made from light-transmissive or semi-light-transmissive materials which can be in colors or transparent or semi-transparent; the control circuit board with illumination device comprises a light source is electrically connected with a power connection unit, the light guide loop is configured at the outer of the thermal conduction device which is inside the shell, hereby, the light source emitting the light in the light guide loop, and evenly disperse lighting effect with or without colors inside the shell through the lighting holes.

7. The aroma and lighting emitting system as claimed in claim 6, said light guide loop including an inner flange, the inner flange formed in the inner of the light guide loop and the thermal conduction device arranged on the inner flange.

8. The aroma and lighting emitting system as claimed in claim 6, wherein comprising an aroma substance container disposed in the light guide loop inside the shell, the aroma substance container made from light-transmissive or semi-light-transmissive materials which can be in colors or transparent or semi-transparent; the light source emitting the light in the light guide loop and evenly disperse lighting with or without colors inside the shell, and the lighting reflects in the aroma substance container producing lighting effect with or without colors through the whole shell and the lighting holes.

9. The aroma and lighting emitting system as claimed in claim 6, said aroma warmer comprising a support loop and multiple tabs formed around a peripheral side of the support loop, the support loop is arranged around the light guide loop when the shell is arranged on the housing and cover the light guide loop and support loop, the inner wall of the shell is removably retained with the multiple tabs of the support loop.

10. The aroma and lighting emitting system as claimed in claim 7, wherein the aroma warmer comprises an insulating unit, the insulating unit disposed on the inner flange of the light guide loop, the thermal conductive device placed on the insulating unit.

11. The aroma and lighting emitting system as claimed in claim 9, wherein aroma warmer further comprises a fixing device set, a transparent lighting case, the transparent lighting case having a third hole; the control circuit board with illumination device having a fourth hole, the hollow heating element having connection hole; the transparent lighting case cover the control circuit board with illumination device for protection; the connection device connects through the connection hole, the fixing device set, the third hole and the fourth hole; the fixing device set supports the bottom side of the hollow heating element.

12. The aroma and lighting emitting system as claimed in claim 11, wherein said connection device is a screw rod; said fixing device set is an anti-loosening nut being threaded onto said screw rod of said connection device and stopped against said hollow heating element.

13. The aroma and lighting emitting system as claimed in claim 1, wherein the hollow heating element can be an annular heating element or similar annular heating element.

14. The aroma and lighting emitting system as claimed in claim 13, wherein the thermal conduction device can be a metal round plate or a similar metal round plate.

15. The aroma and lighting emitting system as claimed in claim 13, wherein the similar annular heating element, a hollow plum-blossom-shaped heating element, or a hollow irregular annular-shaped heating element.

16. The aroma and lighting emitting system as claimed in claim 14, the thermal conduction device is a metal sheet or an iron sheet.

17. The aroma and lighting emitting system as claimed in claim 1, the aromatic substance including scented wax, aromatic wax, scented candles, wax melts, fragrance blends or essential oil.
